(19) United States
(12) Patent Application Publication
(10) Pub. No.: US 2025/0268517 A1
(43) Pub. Date: Aug. 28, 2025

(54) RADIOGRAPHIC DISCERNABLE SENSORS AND ORTHOPEDIC APPLICATIONS FOR SAME

(71) Applicants: CLEMSON UNIVERSITY, CLEMSON, SC (US); Aravis BioTech LLC, Greenville, SC (US)

(72) Inventors: Jeffrey N. Anker, CLEMSON, SC (US); John David DesJardins, CLEMSON, SC (US); Josh Finkel, San Juan, PR (US)

(21) Appl. No.: 19/061,147

(22) Filed: Feb. 24, 2025

Related U.S. Application Data
(60) Provisional application No. 63/556,722, filed on Feb. 22, 2024.

(51) Int. Cl. A61B 5/00; A61B 6/50

(52) U.S. Cl. CPC A61B 5/4504; A61B 5/6812; A61B 6/505; A61B 2562/0261

(57) ABSTRACT

The present disclosure is generally directed to strain sensors which can be used by clinicians to determine the extent to which a tissue has healed. The strain sensor may comprise a proximal end and a distal end, and may be installed along a tissue, wherein the tissue comprises a break disposed between the distal end and proximal end of the strain sensor.

### CROSS REFERENCE TO RELATED APPLICATION

This application claims filing benefit of U.S. Provisional Patent Application Ser. No. 63/556,722 having a filing date of Feb. 22, 2024, which is incorporated herein by reference in its entirety.

### STATEMENT REGARDING FEDERALLY SPONSORED RESEARCH OR DEVELOPMENT

This invention was made with government support under Grant No. R43AR081748 awarded by the National Institutes of Health. The government has certain rights in the invention.

### BACKGROUND

Over 28 million musculoskeletal injuries are treated annually in the U.S. including 2 million fracture fixation surgeries. Of these, tibia fractures are the most common long bone fracture. Unfortunately, such fractures are frequently associated with complications (delayed union, non-union, and infection), particularly for severe trauma such as is often sustained in combat. For instance, non-union is a significant complication (approximately 100,000 injuries, 5% of all fixation surgeries in the U.S. go on to non-union), with even higher rates for severe trauma. Infection at the site of orthopedic surgery is an on-going issue and, while the incidence has been reduced due to improvements in both surgical and post-operative procedures, its prevalence is still unacceptably high. Such complications can lead to long-term or even permanent disability or death and are responsible for significant direct and indirect health care costs.

While a variety of supporting implants and adjunct therapies are available to care givers, a crucial issue leading to complication is the inability to directly evaluate health and healing of the local area. Physicians routinely acquire X-ray images as part of diagnosis and evaluation and while these images can show the hardware and fracture callus, they do not measure mechanical properties of the fracture and cannot detect early-stage infection. 3-dimensional CT images are better at indicating bone density and determining if union has occurred, but are expensive, expose the patient to significant radiation doses (typically around 300× more than a standard X-ray), and are imperfect, especially when allografts are used. For some fracture types, dynamic X-ray images can be acquired to measure bone motion with and without external load in order to assess fracture stability. In practice however this is highly challenging. For example, even in spine fusion, where spinal processes are clearly evident and can move significantly, it has been determined that inter-observer variation of spinous process movement can be about 1.5 mm, with differences as large as 3.5 mm, compared to a recommended bone fusion cutoff criteria of less than 1 mm.

When considering localized infection, external visualization techniques provide little or no clues, particular in early stages of infection. Unfortunately, infection at orthopedic trauma sites are generally not diagnosed until after the infection has spread and symptoms have become systemic.

The lack of widely applicable tests to assess bone health such as load bearing state and early-stage infection presents a major challenge for physicians and patients. Infection at implant sites can require additional surgery or even become life-threatening when diagnosis is delayed. Weight bearing before the fracture callus is sufficiently strong carries risk of refracture and/or hardware failure. On the other hand, unnecessary delay in weight bearing can hamper rehabilitation and is highly costly in terms of lost days of activity. Studies have shown that when the fractured bone has at least 25% of the bending stiffness of intact bone, weight bearing rarely leads to refracture or hardware failure. For externally fixed devices, percutaneous pins can be directly loaded to assess stiffness. When testing is carried out and this 25% threshold is used, the majority of patients had external device removal an average of 2.3 weeks earlier without refracture. Load testing on externally fixated devices can likewise identify patients with delayed and non-union for weight bearing restrictions and additional interventions.

Most orthopedic surgeries involve internal fixation, which require either a percutaneously connected gauge or remote measurements to assess load bearing capabilities during healing. A percutaneously connected strain gauge is impractical for patients and presents a number of safety challenges. A variety of remote interrogation methods based upon implanted wireless devices, ultrasound, vibrational analysis, and other approaches have been examined for non-invasive measurement of strain on orthopedic implants, but these generally require significant development as well as equipment and/or expertise currently unavailable to most care givers.

What are needed in the art are passive implantable sensors for use in conjunction with orthopedic implants that can be easily read by conventional non-invasive methods to assess local conditions and bone health at the local site. In particular, what are needed are passive sensors capable of assessing strain under load and/or early-stage signs of infection and thereby to determine a current state of bone health. For instance, a passive sensor locatable on bone fixation devices that can assess health and healing in the local area of an orthopedic implant by use of conventional radiography methods would be of great benefit.

### SUMMARY

According to one embodiment disclosed herein is a strain sensor comprising a pickup arm comprising a free end and a static end, wherein the static end is directly or indirectly coupled to an orthopedic implant or orthopedic tissue, a gain mechanism comprising a free end and a static end, wherein the static end of the gain mechanism is directly or indirectly coupled to the orthopedic implant or orthopedic tissue and wherein the free end of the gain mechanism and the free end of the pickup arm are operatively coupled.

### BRIEF DESCRIPTION OF THE FIGURES

A full and enabling disclosure of the present subject matter, including the best mode thereof to one of ordinary skill in the art, is set forth more particularly in the remainder of the specification, including reference to the accompanying figures in which:

FIGS. 1A and 1B depict a chemical sensor of the present disclosure;

FIG. 2 depicts a side view chemical sensor cartridge and casing of the present disclosure;

FIGS. 3A-3F depict isometric views of barbs of the present disclosure;

FIGS. 4A and 4B depict isometric views of barbs of the present disclosure;

FIGS. 5A and 5B depict a chemical sensor of the present disclosure having a collar housing;

FIGS. 6A and 6B depict a chemical sensor of the present disclosure from a side perspective;

FIGS. 7A and 7B depict chemical sensors of the present disclosure having a snap-ring housing;

FIGS. 8A and 8B depict a chemical sensor of the present disclosure fitted to an orthopedic implant;

FIGS. 9A-9D depicts a gain mechanism of the present disclosure;

FIG. 10 is an isometric view of a cartridge coupled to a gain mechanism of the present disclosure;

FIGS. 11A and 11B are depictions of a chemical sensor of the present disclosure;

FIGS. 12A and 12B are depictions of a chemical sensor of the present disclosure;

FIGS. 13A, 13B and 13C are graphs showing the maintained sensitivity of the chemical sensor to pH before and after sterilization via autoclaving (13A), ETO sterilization (13B) and gamma sterilization (13C);

FIG. 14 is a graph showing the responsiveness of a chemical sensor of the present disclosure across a domain of pH 5.5 to 8 before and after sterilization using autoclaving, ETO sterilization and gamma sterilization;

FIG. 15 shows of a chemical sensor of the present disclosure mounted to the exterior of a dynamic hip screw

FIG. 16 shows an orthopedic implant comprising a chemical sensor of the present disclosure;

FIG. 17 shows an orthopedic implant comprising a chemical sensor of the present disclosure;

FIG. 18 shows a lag screw which may be used in a dynamic hip screw, with a strain sensor disposed therein;

FIGS. 19A-19D depict a strain sensor of the present disclosure;

FIGS. 20A and 20B depict a strain sensor comprising a geared gain mechanism;

FIG. 21 depicts the geared gain mechanism of FIGS. 20A and 20B;

FIGS. 22A-22C depict a strain sensor of the present disclosure;

FIGS. 23A and 23B depict a strain sensor of the present disclosure;

FIG. 24 depicts an orthopedic implant comprising a strain sensor of the present disclosure;

FIGS. 25A and 25B depict a strain sensor of the present disclosure, wherein the strain sensor comprises a single-arm scissor mechanism;

FIGS. 26A and 26B depict a strain sensor of the present disclosure, wherein the strain sensor is enclosed by a housing comprising radiopaque marks;

FIGS. 27A and 27B depict a strain sensor of the present disclosure; and

FIG. 28 depicts a lag screw with a strain sensor of the present disclosure disposed therein.

Repeat use of reference characters in the present specification and drawings is intended to represent the same or analogous features or elements of the present invention.

### DETAILED DESCRIPTION

Reference will now be made in detail to various embodiments of the disclosed subject matter, one or more examples of which are set forth below. Each embodiment is provided by way of explanation of the subject matter, not limitation thereof. In fact, it will be apparent to those skilled in the art that various modifications and variations may be made in the present disclosure without departing from the scope or spirit of the subject matter. For instance, features illustrated or described as part of one embodiment may be used in another embodiment to yield a still further embodiment.

In general, the present disclosure is directed to devices for determining bone health. More specifically, disclosed are sensors that can be utilized in conjunction with orthopedic implants that can provide information with regard to the ability of orthopedic tissue (e.g., bones such as the tibia, femur, fibula, ulna, ligaments, tendons, joints such as hips, shoulders, elbows, etc.) to support a load and/or the presence of infection or other health issue in the area of an implant. Beneficially, the sensors are passive and can be read by use of standard radiographic imaging techniques (e.g., X-ray, computed tomography (CT) scanning, etc.) that are already taken as part of a standard patient evaluation. Moreover, the sensors can be affixed to known implantable support devices (e.g., tibial plates, spinal inserts, screws, rods, pins, etc.) without excessive modification of the implants so as to non-invasively monitor the implant to provide a quantitative assessment of one or more characteristics in the local area of the implant.

The sensors of the present disclosure have several advantages including, but not limited to, ease of use for clinicians, standardization, sterilizability and modularity. Additionally, the sensors of the present disclosure may be manufactured prior to implantation, reducing the extent to which a clinician may need to provide a bespoke solution which may lack in standardization and/or quality.

In one embodiment, a sensor can be a chemical sensor that can be utilized to assess the local area of an implant for one or more analytes. For instance, an area can be assessed for the presence of an analyte that can indicate early-stage infection at the implant site.

According to this embodiment, the sensor can include a material that in the presence of a targeted analyte can exhibit a change that is detectable by radiographic imaging. For example, the sensor can exhibit a dimensional change upon interaction with the targeted analyte. The sensor can also include a scale, and optionally, a radiographically discernable component such as an extended rod, channel, or the like that can amplify and improve visualization of the dimensional change of the sensor in the presence of the analyte.

In general, an analyte-sensitive sensor can include a hydrogel portion that is chemically configured to vary its displacement volume according to changes in concentration of an analyte in the surrounding area. Hydrogels sensitive to analytes have been described, for instance in U.S. Pat. No. 6,751,491 to Lew, et al., U.S. Pat. No. 6,835,553 to In Suk Han, et al., U.S. Patent Application Publication No. 2009/0170209 to Machauf, et al., and U.S. Pat. Nos. 10,667,745 and 11,684,307 to Anker, et al., all of which are incorporated herein by reference.

An analyte-sensitive hydrogel may include any analyte-sensitive material that can directly or indirectly modify the displacement volume of the hydrogel in response to a change in analyte concentration. For example, the analyte-sensitive material can directly bond with the analyte to modify the displacement volume of the hydrogel. Alternatively, the analyte-sensitive material can catalyze a reaction of the analyte and the reaction product can directly or indirectly modify the displacement volume of the hydrogel.

By way of example and without limitation, in one embodiment, an analyte-sensitive material can be immobilized within a pH-sensitive hydrogel. As utilized herein, the term ‘pH-sensitive hydrogel’ refers generally to a hydrogel modified to contain pendant charged groups on polymers of the hydrogel in proportions that produce an overall acidic or basic environment in the fluid within the gel. An analyte-sensitive material present in the hydrogel can bind to the targeted analyte or catalyze a reaction of the targeted analyte and thereby provide a charged product within the hydrogel. The charged product generated by activity of the analyte-sensitive material can cause the hydrogel to change its displacement volume (swell or shrink).

A pH-sensitive hydrogel can include a copolymer synthesized from various types of methacrylate-derived monomers by free radical solution polymerization as are known in the art. Such copolymers can be relatively tough, flexible polymers that are highly biocompatible and inert yet non-degradable in vivo. Synthesis conditions for pH-sensitive hydrogels have been well established. In one particular embodiment acrylamide or dimethylacrylamide can be utilized as a polymer backbone, sodium acrylate as a pH-sensitive pendant group, and N,N′-methylene-bis-acrylamide as the cross linker. To obtain pH sensitive hydrogel copolymers with desired properties, the ratios of the monomers and cross-linker can be varied as is known in the art.

The targeted analyte can include one or more compounds the presence of which in the local area of an orthopedic implant can indicate infection. For instance, the analyte can include a pathogen or a determinant of a pathogen that can be, e.g. an endotoxin or exotoxin of the pathogen or a compound present in the local area due to an infection response of the host. Analytes of interest can include, without limitation, hydrogen ions (e.g., pH), quorum sensing molecules, glucose, lactic acid, carbon dioxide, proteins, proteases, infection-indicating peptides such as alpha-defensins, cytokines, enzymes such as leukocyte esterase, antibiotics, drugs, etc.

In one particular embodiment, the targeted analyte of a sensor can be glucose, which can provide an indication of infection from a broad range of causative agents. Even at early stages, the presence of infection is generally accompanied by an inflammatory state. Cytokines released during inflammation can affect glucose metabolism directly or indirectly by augmenting glucoregulatory hormone secretion and curtailing insulin release. In particular, glucose can be shunted away from its normal sites of utilization to sites that assist in overcoming infection, and as such increased levels of glucose in a local area can be an excellent broad-spectrum indicator of early-stage infection in the area.

One such glucose-sensitive chemical sensor can include an immobilized glucose oxidase that can detect the presence of glucose via the conversion of glucose to gluconic acid by the enzyme. The rate of gluconic acid formation is proportional to the glucose concentration in the hydrogel at the reaction location. A change in glucose concentration in the fluid surrounding the hydrogel can lead to a change in the pH value within the hydrogel due to the glucose oxidase-catalyzed production of gluconic acid. In particular, the gluconic acid product can protonate pH-sensitive pendant groups in the hydrogel and causes the hydrogel to swell or shrink, depending on the nature of the pendant groups. If the hydrogel contains basic pendant groups such as diethylaminoethyl methacrylate, it will swell when pH decreases. If it contains acidic pendant groups such as acrylic acid (AA), the hydrogel will shrink when pH decreases.

In another embodiment, an analyte-sensitive material can directly interact with the targeted analyte. For instance, a hydrogel can include analyte binding molecules and upon binding, the hydrogel can exhibit a change in volume due to steric changes within the hydrogel or the presence of charge groups on the bound analyte (e.g., in the case of a pH sensitive hydrogel).

In yet another embodiment, an analyte-sensitive hydrogel can include analyte binding molecules and analyte analogue molecules immobilized therein, optionally in conjunction with charged pendant groups on the hydrogel polymer. In the absence of free analyte, the immobilized analyte binding molecules can bind to the immobilized analyte analogue molecules, forming what are in effect crosslinks within the hydrogel (generally non-covalent crosslinks). As free analyte in the local area interacts with the hydrogel, binding competition can displace immobilized analyte analogue molecules with free analyte molecules, thus reducing the number of crosslinks. This reduction in crosslinking causes swelling of the hydrogel. This type of sensitive hydrogel is based on a competitive mechanism and does not require oxygen-consuming enzymatic reactions.

In one embodiment, an analyte binding hydrogel can contain immobilized phenylboronic acid or another glucose binding molecule that can be covalently bonded to the hydrogel polymer, for instance via diols on the hydrogel polymer. For instance, a hydrogel can include one or more polyols such as poly(vinyl alcohol) (PVA), with other pendant groups as necessary to achieve the desired sensitivity, response, and durability. As discussed above, an analyte binding hydrogel can optionally include analyte analogue molecules, such as a D-sugar or other carbohydrate that can bind the glucose binding molecule with a non-covalent bond. The volume of this hydrogel can change in the presence of free glucose due to a competitive binding effect of the free glucose with the glucose binding molecule in place of the glucose analogue molecule. When glucose concentration increases near the implant area, additional amounts of free glucose can diffuse into the hydrogel and displace the glucose analogue (e.g., a D-sugar) from the binding sites of the immobilized glucose binding molecule. This can reduce the hydrogel crosslink density, and thus the hydrogel can swell.

To produce a competitive binding glucose targeting hydrogel, glucose analogue molecule-conjugated and glucose binding molecule-conjugated vinyl monomers can be synthesized. These conjugated monomers can then be co-polymerized with cross linkers and either cationic or anionic monomers as discussed above. The cross linker introduces a small number of permanent crosslinks into the hydrogel in order to keep hydrogel integrity at all free glucose concentrations.

Examples of glucose binding molecules can include, without limitation, lectins (e.g., Con A, glucokinase, xylose isomerase, and isolactin I), glucose antibody, concanavalin A, boronic acid, thiols, cell membrane receptors, cytosol receptors, nuclear receptors, heparin, DNA, RNA, polylysine, polyarginine triazine dye, commassie blue, azure A, metal binding molecules including chelating agents, etc. Examples of glucose analogue molecules can include, without limitation, glucose antigen, glucose cofactor, glucose substrate, glucose inhibitor, D-sugar, carbohydrates, 1,2-cis-diol sugars, cysteine, metal ions (e.g., Ca, Mg, etc.), etc.

A sensor can also include a semi-permeable membrane that can protect the hydrogel such that analyte-containing body fluid is free to pass through the membrane and permeate the hydrogel. A membrane can generally have a thickness on the order of microns. In one embodiment, a membrane can be radiographically transparent. In addition, the membrane can be biocompatible and have sufficient stiffness to substantially prevent the hydrogel from swelling in undesirable directions and from escaping into the surrounding area.

In one embodiment, a semipermeable membrane can be permeable to the passage of the analyte and any other materials utilized in the sensing application (e.g., glucose, oxygen, carbon dioxide, lactic acid, gluconic acid, etc.) and can be totally or substantially impermeable to the passage of blood clots, cells, and non-analyte proteins. In addition, a semipermeable membrane can be an inert, nontoxic material that maintains its integrity when implanted in humans. A suitable biocompatible semipermeable material, to minimize immune reactions and to prevent protein and cell absorption, can be selected from the group of polymers including, without limitation, cellulose acetate, methyl cellulose, polyvinyl alcohol, polypropylene, HEMA, tetra-acrylated poly(ethylene glycol) (PEG), and/or polyurethane. For example, thin hydrophobic membranes such as PDMS or parafilm can serve as gas permeable membranes while excluding water-soluble ions and molecules. Similarly, hydrogels, commercially available dialysis membranes, and filters, can be used to molecules smaller than the pore size, while excluding molecules (as well as cells and tissues) larger than the pore size.

There is an extensive body of knowledge on developing stimuli-responsive gels for chemical sensors, actuators (e.g. artificial muscles), and drug release materials. For example, a partial review of stimuli responsive materials is given in Journal of Controlled Release 190 (2014) 337-351. Many methods are available to detect different types of analytes include coupling pH-responsive gels to enzymes that generate acidic products (e.g. glucose and oxygen can be detected by encapsulating glucose oxidase and catalase into a pH responsive gel to produce gluconic acid according to the concentration of glucose and oxygen present). Alternatively, molecular recognition interactions (e.g. antibody-antigen, lectin-carbohydrate, ionophore-ion, enzyme-substrate, DNA-complementary DNA, aptamer-substrate, etc.) can be used to affect gel swelling. The gel swelling can be measured based upon axial expansion, bending of a multilayered structure with different expansion properties in the structure (e.g. in cantilever-based chemical sensors, and similar to the working of bimetallic thermal sensors/actuators and paper-based hygrometers). Multi-layered structures can be formed by bonding two or more structures together, or allowing particles to settle within a polymer, varying the amount of cross-linking in the top of a polymer film compared with the bottom, etc. Multiple sensors can be used to increase specificity and account for interactions between multiple variables. For example, the combination of a pH sensor and a separate glucose sensor with glucose oxidase can be used to account for how the ambient pH affects the glucose sensor in order to estimate the two analyte concentrations. In addition, measuring multiple analytes may be more indicative of pathology (e.g. infection) than a single analyte.

Of course, additional sensitive materials specific to other analytes of interest can also be included in a chemical sensor, and a sensor is in no way intended to be limited to determination of the presence or concentration of any particular number of analytes. Moreover, multiple sensors including any combination of chemical and strain sensors can be used in an area. For instance, a joint, bone, or general area including one or more orthopedic tissues can include one or more strain sensors optionally in conjunction with one or more chemical sensors. Beneficially, the sensors are passive, and as such require no internal energy source or active interrogation for examination. The sensors can be easily read with standard radiography available in all hospitals and many clinics and can be incorporated into existing implant constructs to provide robust, durable performance for use in conjunction with any orthopedic tissue.

The chemical sensor may comprise a casing, wherein a cartridge may be disposed, an optional gain mechanism and a housing. A cartridge may comprise a hydrogel as described above, in addition to a barb which can slide along an optional internal rail. The casing may comprise, in embodiments, a scale comprising radiopaque markers, an aperture to allow in-flow of an analyte to the hydrogel, and optional holes for pinning the barb and/or hydrogel to the casing.

The barb can be used to pin the cartridge to the casing, thereby securing the hydrogel in place. The barb can additionally comprise a radiopaque marker. The barb can serve several purposes, including holding radiodense markers to show hydrogel position, reproducibly positioning the hydrogel within the casing during loading of the cartridge in the casing, and serving as a sliding surface against the casing to allow expansion in a proscribed direction without direct contact between the hydrogel and casing. The barb may have a variety of geometries including with a cylindrical cross-section in a larger concentric cylindrical casing hole. This shape is not limiting provided that the barb may slide in the hole. Additionally, the barb can have a hole in it, through which an internal rail can fit and slide. The presence of such an internal rail can allow the barb to slide only along the longitudinal axis of the cartridge despite multidimensional swelling or contraction of the hydrogel. Alternatively, the casing or cartridge may comprise an exterior sliding surface or compliant mechanism which can constrain the expansion of the hydrogel to one dimension.

The casing may include a semipermeable membrane placed to prevent contact between the surrounding environment and hydrogel or barb, while allowing the analyte to pass through the membrane and interact with the analyte-sensitive hydrogel. The casing may further comprise an aperture which can allow the cartridge to be pinned to the casing. The casing and membrane may also be configured to prevent surrounding tissue from mechanically pressing on the cartridge. As an example, a dialysis membrane with pore size larger than the analyte may be placed around the outside of the casing to block large proteins, cells, and tissue from entering into the mechanism. Other embodiments are also possible, including integrating the semipermeable membrane into the casing openings.

The casing and housing, may be fashioned out of materials including, but not limited to, polyether ether ketone, polyethylene, polyvinyl alcohol, or titanium. In general, the invention is practicable with a housing material which is biocompatible, has good lubricity, has a moderate to high degree of radiolucence, is easily machinable, and is not likely to foul.

It should be understood that the hydrogel motion need not be confined to linear expansion, and that other modes may also be employed. For example, bilayer materials, where one layer expands more than another are commonly used to make bending-based sensors, including in widely used analog bilayer thermometers and humidity gauges.

The chemical sensor of the present disclosure is not particularly limited in its methods of employment or use. However, a method may comprise forming placing a barb, an optional internal rail, and an optional mounting screw within a mold. The mold may then be infiltrated with the hydrogel precursor, which after reaction, serves to form a completed cartridge. Said cartridge can be loaded into the casing of a chemical sensor either in advance of a surgical operation, or at the time of a surgical operation. The cartridge may be placed and secured within the casing of the chemical sensor by pinning the hydrogel to the casing through pinning holes. Alternatively, the barb may be secured and/or aligned within the casing using holes in the barb and/or casing. Further, cartridge may comprise a mounting screw, which may be used screw the cartridge into the casing.

For instance, a system comprising an orthopedic implant and an accompanying chemical sensor that can be provided to clinicians can be envisioned. As will be discussed later, the presently described hydrogels and chemical sensor employing said hydrogels are fully sterilizable. Thus, a kit may comprise an orthopedic implant, a chemical sensor and optional installation hardware.

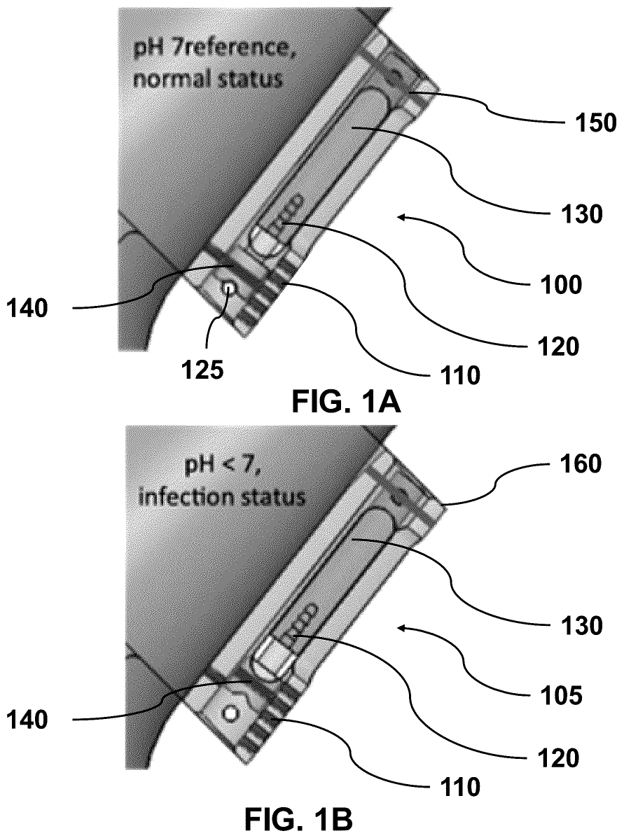

The present disclosure may be understood with further reference to the figures. An example of a chemical sensor as it may be used may be found in FIGS. 1A and 1B. As shown in FIG. 1A, the chemical sensor 100 comprises a cartridge comprising a radiopaque marker 140, a barb 120 comprising a barb hole 125, a hydrogel 130, wherein at least a portion of the barb 120 is encompassed by the hydrogel 130, and a pinning rod 150 which may secure the cartridge inside the casing 160. Further, the sensor comprises, on the casing 160, reference markers 110. As the pH of the matrix surrounding the sensor 100 falls below 7 (generally indicative of infection), the hydrogel may change in size, such as through shrinking. FIG. 1B shows the hydrogel's decrease in volume, as can be determined by the relative displacement of the radiopaque marker 140 in FIGS. 1A and 1B.

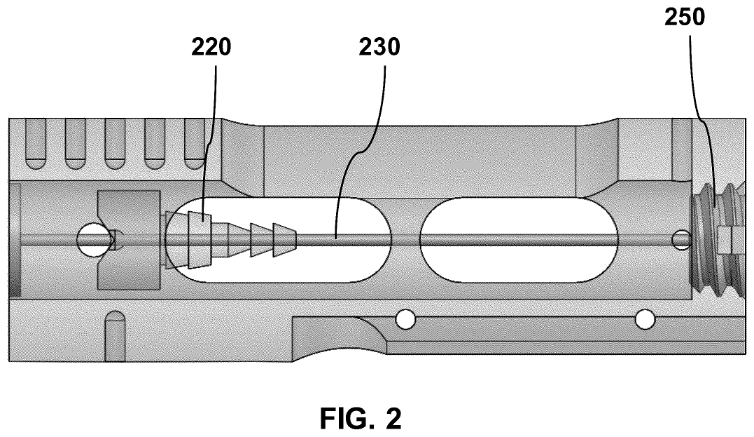

FIG. 2 is a side view of a cartridge and casing of the present disclosure. In addition to the features of FIGS. 1A and 1B, the cartridge of FIG. 2 comprises internal rail 230, wherein barb 220 may slide along internal rail 230, and positioning screw 250.

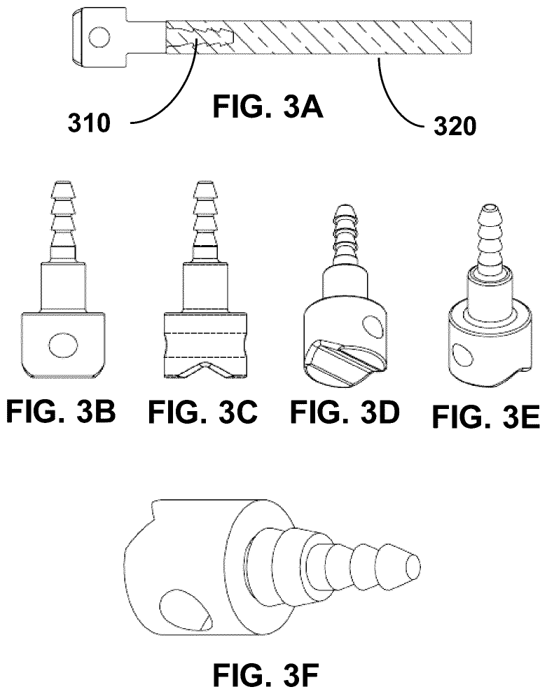

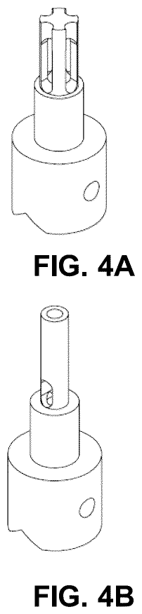

Turning now to the cartridge, FIG. 3A shows a cartridge comprising the hydrogel 620 encompassing at least a portion of barb 310. Further, FIGS. 3B-3F provide various views of exemplary barbs. Said barbs may comprise a radiopaque marker that can be visualized under, but not limited to, X-ray. FIGS. 4A and 4B meanwhile show alternate designs for barbs that fall within the scope of the present disclosure.

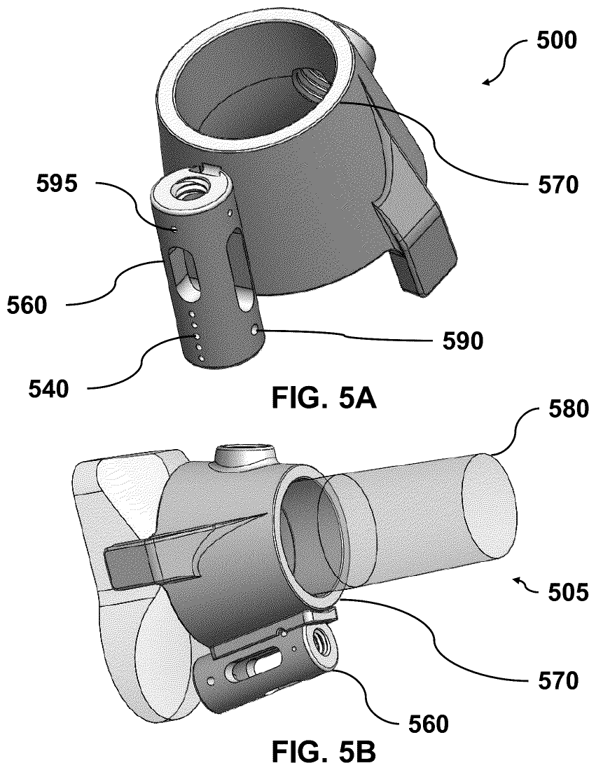

FIGS. 5A and 5B are isometric views of one embodiment of a chemical sensor described by the present disclosure. As shown in FIG. 5A, the chemical sensor 500 may comprise, as discussed, a housing 570 coupled to a casing 560, into which a cartridge may be placed. Said casing 560 may comprise radiopaque markers 540 and barb holes 590 and cartridge holes 595, both of which may be used alone or in combination to pin the cartridge to the casing 560. Further, housing 570 may comprise a shape optimal for fitment to a biological tissue, such as a bone. As shown in FIG. 5B, the chemical sensor 505 may be placed over a portion of a bone or orthopedic device 580. It is within the scope of the present disclosure, however, that housing 570 may comprise a different shape depending on the particular circumstance that a clinician may encounter. For instance, the housing 570 may be in the form of a snap-ring, a collar or a clamp, or other means of non-permanently marking the surface of bone or orthopedic implant 580. In other embodiments, the housing 570 may comprise a plate and screw system.

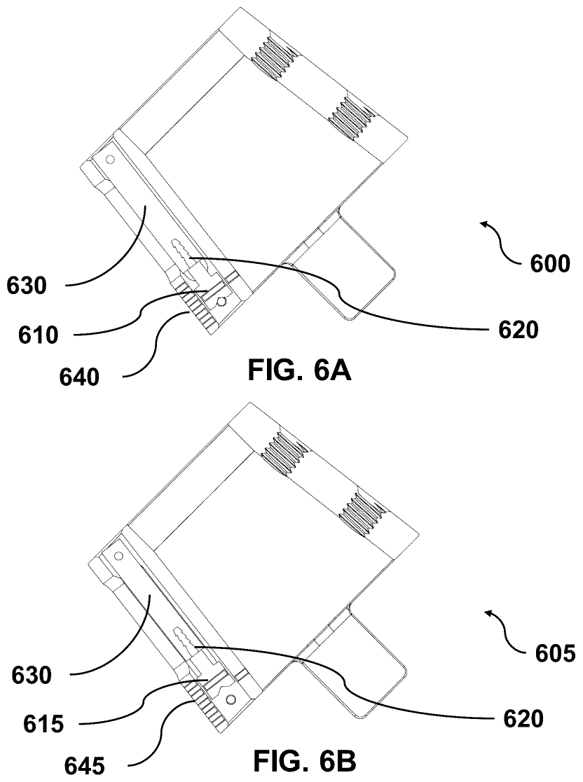

A cross-sectional view of chemical sensor 600 may be seen in FIG. 6A. As stated above, the cartridge may comprise a hydrogel 630 with a barb 620 at least partially encased therein. Further, the cartridge may further comprise a radiopaque marker 610, the location of which may be non-invasively probed, such as through X-ray, and compared to the location of reference radiopaque markers 640. For instance, as shown in FIG. 6B, radiopaque marker 615 has moved in location relative to reference radiopaque markers 645 as may be compared to in FIG. 6A. Further, FIGS. 6A and 6B provide isometric and side views of medical devices 600, 605 comprising a chemical sensor 620 in contact with bone or orthopedic device 630.

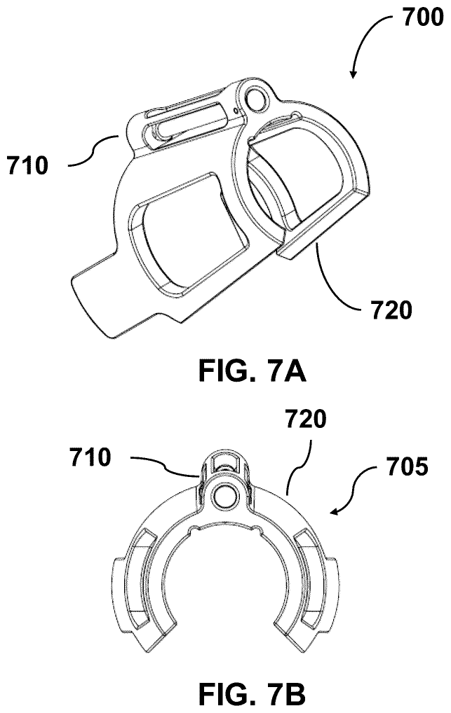

While FIGS. 5A and 5B may show the chemical sensor comprising a housing comprising a closed collar, the present disclosure is not so limited. FIGS. 7A and 7B show isometric and head-on views, respectively, of chemical sensors 700, 705 comprising the casing 710 as described above mechanically coupled with a housing 720 which comprising a snap-ring. Said housing 720 may provide advantages in situations including, but not limited to, where a collar may not be able to slide over a biological tissue.

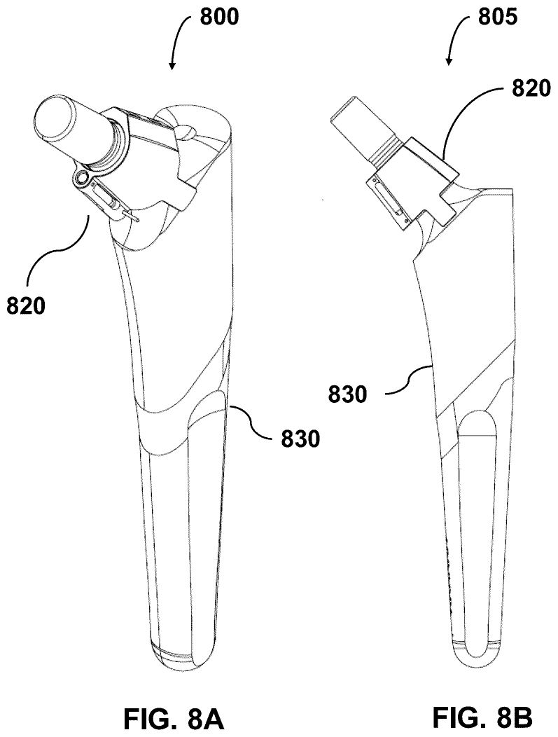

FIGS. 8A and 8B show an orthopedic implant with a chemical sensor 800, 805. As shown, an orthopedic implant 830 may be fitted with a chemical sensor 820. The collar of chemical sensor 820 may serve to secure the chemical sensor around a feature of the orthopedic implant 830, in this case said feature comprising a neck.

While the provision of a chemical sensor as described thus far contributes to the art at least in terms of standardization and repeatability, as well as ease of use, the present disclosure further contemplates means for making clinical determinations of tissue health more facile than may be possible through the use of a hydrogel alone. Thus, the present disclosure will now describe several gain mechanisms, though the present disclosure is not so limited to those described herein. Such gain mechanisms allow for a minimal change in a quantity, such as the volume of a hydrogel, to be magnified while maintaining a high dynamic range. The gain mechanism may be formed as part of the cartridge, or it may be present as part of the casing into which the cartridge may be inserted. In the case that the gain mechanism is formed as part of the casing, the gain mechanism and the cartridge may comprise a linkage.

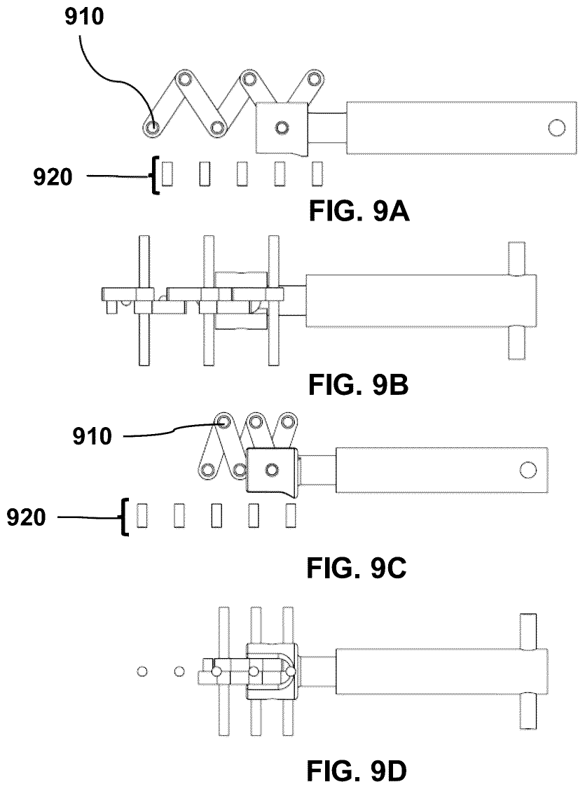

Examples of said gain mechanisms may be found in FIGS. 9A-12B and 19A-23B and 25A-27B. While the latter group of gain mechanisms may be described in the context of a following embodiment, it is within the skill of one in the art to adapt such mechanisms for use with a hydrogel.

As can be seen in FIGS. 9A-D, the gain mechanism may comprise a single-arm scissor mechanism. Said gain mechanism may further comprise radiopaque markers 910, the position of which may be compared with reference radiopaque markers 920.

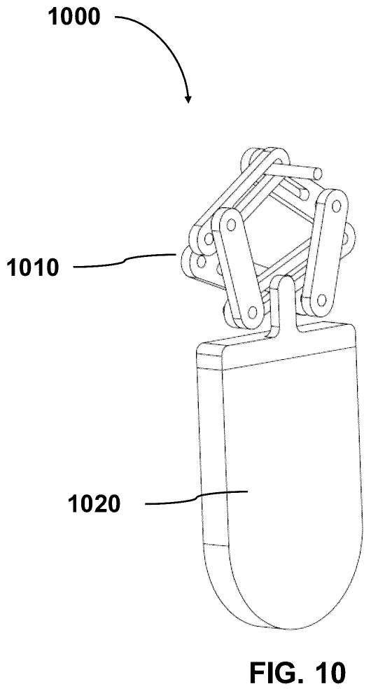

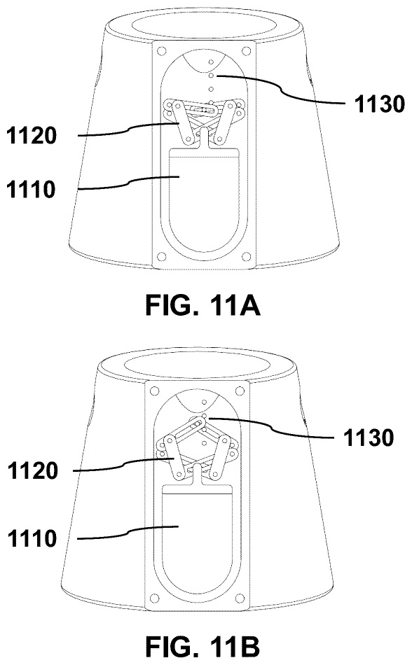

FIG. 10 shows a hydrogel 1020 coupled to a gain mechanism 1010. FIGS. 11A and 11B show that a small decrease in volume of hydrogel 1110 leads to a marked change in the conformation of gain mechanism 1120. The location of the radiopaque markers on gain mechanism 1120 may be compared to that of reference radiopaque markers 1130.

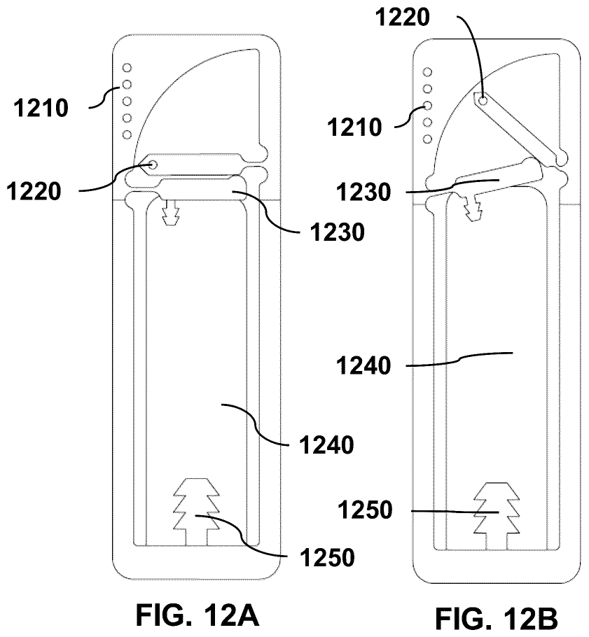

FIGS. 12A and 12B show another gain mechanism within the scope of the present disclosure. Said gain mechanism operates by comparing the location of radiopaque marker 1220 to that of reference radiopaque markers 1210 before and after an analyte has been introduced to the hydrogel 1240. Said hydrogel 1240 is secured in place by barb 1250, and exerts force via expansion on gain mechanism 1230, thereby causing radiopaque marker 1220 to move.

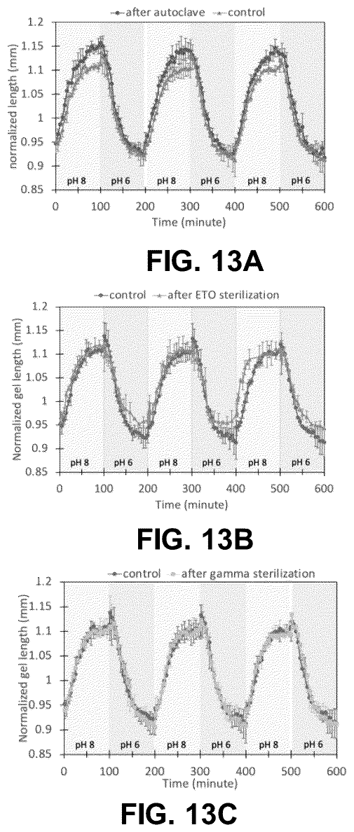

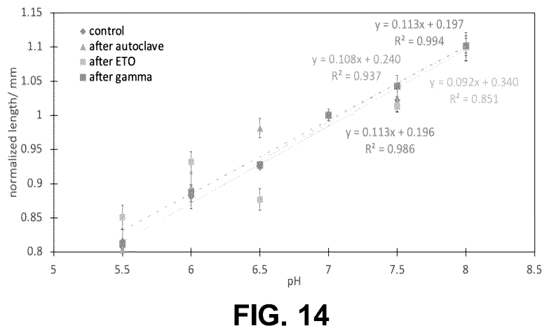

FIGS. 13A-13C showcase the ability of the presently described chemical sensor to undergo repeated pH cycling with little to no loss of responsiveness after autoclaving (FIG. 13A), ETO sterilization (FIG. 13B), and gamma sterilization (FIG. 13C) as compared to a control. FIG. 14 shows the responsiveness of the chemical sensor across a wide pH range (5.5 to 8) after sterilization by any of the three methods mentioned above.

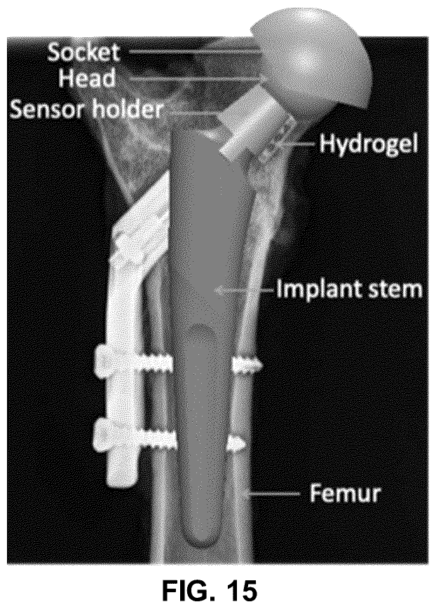

As a non-limiting example of use of such a chemical sensor, FIG. 15 shows an orthopedic device comprising the chemical sensor of the present disclosure.

Thus, while the orthopedic device comprising a chemical sensor shown in FIG. 15 may dispose the chemical sensor within joint of a hip, it is within the scope of the present disclosure to use the chemical sensor in other physiological locations. For instance, sensors can be incorporated with fixation devices in a wide variety of orthopedic applications in addition to long bone fixation applications including, without limitation, spine fusion, hip fixation, total or partial joint replacement of any joint, and others to provide mechanical and mechanically transduced orthopedic measurements. Implantable orthopedic components of interest include, without limitation, cervical spine plates, spinal fusion devices, dynamic hip screws, compression plates, locking plates, screws, intramedullary rods, etc. A chemical sensor can be affixed to an orthopedic implant or bone, but this is not a requirement of a chemical sensor, as the response of the chemical sensor does not depend upon the action of the implant or the bone. Accordingly, the chemical sensor need not be directly or indirectly attached to the bone at any point but should be implantable in the local area of the implant. As such, it may be convenient to locate a chemical sensor on the implant. For instance, in some embodiments, the chemical sensor may comprise a housing that can attach to an orthopedic implant by a variety of means including, but not limited to, a pressure fit, a snap fit or a keyed mechanism. Further, the orthopedic implant may be designed with a mechanism designed to retain the chemical sensor.

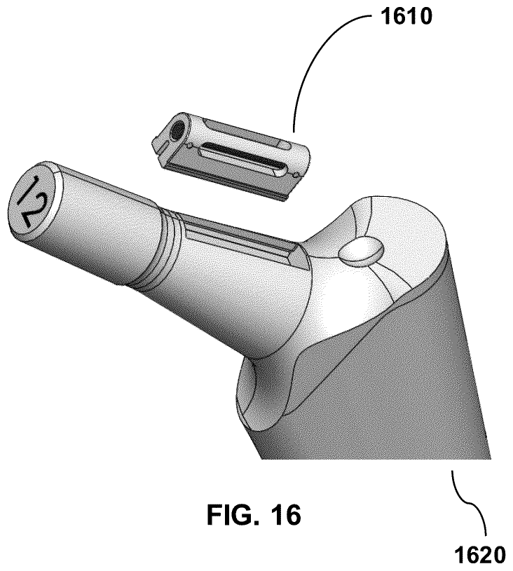

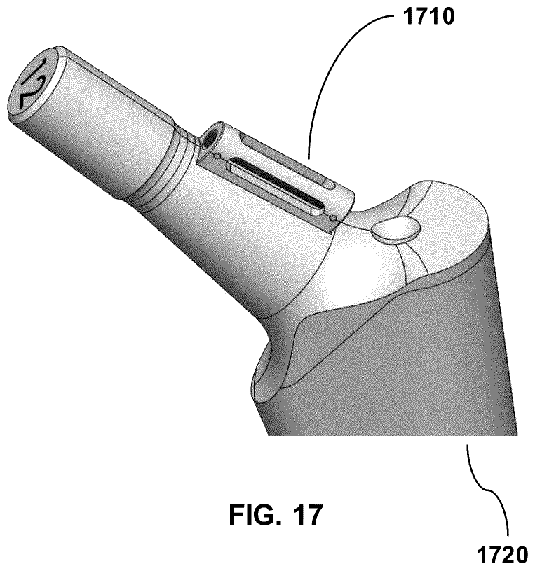

As an example of an alternate fitment, FIGS. 16 and 17 show a chemical sensor 1610, 1710 being snap-fit into an orthopedic implant 1620, 1720.

Alternatively, a chemical sensor can be located directly on a bone or on another structure in the general area. In any case, a chemical sensor can be located such that it can be examined radiographically to determine the presence of an analyte in the local area of the implant. In one embodiment, the analyte-sensitive material can function as the radiographically discernable material.

In embodiments, the chemical sensor may be mechanically linked to an orthopedic implant or an orthopedic tissue such that the chemical sensor is disposed within the synovial fluid of a joint, such as the joint of a knee, hip, elbow, shoulder or others. In embodiments, the chemical sensor may be mechanically linked to an orthopedic implant or an orthopedic tissue such that the chemical sensor is disposed within the synovial fluid of a joint, such as the joint of a knee, hip, elbow, shoulder or others. The chemical sensor, therefore, may be provided to a clinician in the form of a kit. Said kit may comprise the chemical sensor, an orthopedic implant and optionally installation hardware. Furthermore, said kit may comprise a container which contains the chemical sensor, orthopedic implant and optional installation hardware. As shown above, the kit may be sterilized without the loss of performance of the chemical sensor.

In another embodiment, the passive sensor can be a strain sensor that can be utilized to determine when a damaged bone, joint, or soft orthopedic tissue is sufficiently healed to allow safe weight-bearing upon the limb. While the instant application discusses utilization of disclosed sensors in conjunction with bones, it should be understood that disclosed sensors can be utilized in conjunction with any orthopedic implant and any orthopedic tissue including, without limitation, bone, ligament, tendon, meniscus, etc. The strain sensors are based upon the transition of load from a supporting implant to the orthopedic tissue (e.g., bone) as the tissue heals and becomes stronger. For instance, at initial fixation, a fractured bone can take little or no load without possibility of further injury. As such, most or all of a load (e.g., an axial load in the case of a long bone) placed on the bone will be carried by the supporting orthopedic implant structure.

Passive strain sensors have been designed around the concept of the load transference from an orthopedic fixation device to the surrounding tissue that takes place during normal healing. The sensors can be utilized to determine the distribution of a load between a bone and a fixation device and as such, can provide an indication of the bone health and level of healing. The passive sensors can be mounted to orthopedic devices so as to non-invasively serve to quantify implant bending/deflection using standard radiography. In some embodiments, the strain sensors can amplify the normal deflection of an orthopedic implant to provide clear indication of the load transfer to an implant. Through use of the sensors, a care giver can identify the point at which a fracture has sufficiently healed so that safe weight bearing and rehabilitation may begin as well as recognize when healing is insufficient for such weight bearing in order to reduce complications from premature weight bearing and for the early detection of possible complications such as fracture nonunion.

A strain sensor may, in embodiments, comprise a pickup arm and a gain mechanism at opposing ends of the strain sensor which are operatively coupled, such as by a pivot mechanism. Each of the gain mechanism and pickup arm may comprise a static end and free end, wherein the static end is directly or indirectly fixed to an orthopedic implant or orthopedic tissue. For instance, a pickup arm may comprise a free end and a static end, wherein the free end is operatively coupled to the pivot. Thus, a torque applied to the static end of the pickup arm may cause a rotation of the free end of the pickup arm at the pivot. Meanwhile, the gain mechanism may be coupled to the pivot by the free end of the gain mechanism and an optional fixed point by the static end of the gain mechanism. The rotation of the pickup arm at the pivot may cause the gain mechanism to undergo a levering effect when it is fixed, thereby amplifying any movement in the gain mechanism. To ensure the pickup arm is stiff enough to transfer load to the gain mechanism, it is advantageous for it to be stiff, which can be achieved using materials with high elastic modulus and stiff geometries. In some embodiments, in order to allow movement within the cannula, the pickup arm may be tapered.

In any case, the sensor components can be located such that they do not interfere with proper function of the implant and such that they do not cause irritation at the implant site. For instance, a sensor can be located at one side of a compression plate and optionally can utilize unused screw holes. In general, a sensor can have a relatively small profile and can either be set within the profile of the associated orthopedic implant or can extend to a small degree beyond the surface of the orthopedic implant. This can be particularly beneficial when considering a sensor for use in conjunction with an implant configured for location deep within tissue.

The sizes and materials of the various components of a sensor can vary depending upon the particular design of the sensor and the orthopedic implant to which the sensor will be attached. When included, a housing of a sensor can be radiographically transparent.

The materials utilized to form a sensor can be chosen in order to clearly discern the relative motion of a portion of the sensor against a scale. In some embodiments, the scale may have finely designed features (e.g. holes) to allow visualization of small motions, for instance by use of a Vernier-type scale based upon moiré fringes. While some embodiments of a strain sensor, or a housing thereof, may employ a scale, such is not particularly required.

The dimensions of the sensor and/or the scale can be suitable so as to provide a radiographically determinable indication of movement of the attached orthopedic device. For instance, a gain mechanism as described above may be employed. Said gain mechanism may be provided in several forms, some of which are described in detail in the proceeding and following paragraphs. However, without wishing to be bound to any particular theory, a find of the present inventors is that a gain mechanism wherein the displacement of a radiopaque marker may be increased may be particularly useful when the displacement of said marker is parallel with the major axis of the sensor itself. For instance, a change in angle between a distal point and proximal point of the strain sensor may cause a gain mechanism coupled thereto to displace a radiopaque marker along the length of the strain sensor. Such a displacement may be referred to as a parallel displacement, and has several benefits including, but not limited to, a high dynamic range with a lack of saturation due to impacting a biological tissue exterior to the strain sensor itself. For instance, upon applicaiton of a force through the pickup arm, the gain mechanism may increase in length by 8× to 20×. The high dynamic range of such a parallel displacement system allows for a clinician, upon radiographic examination, to make a quick, binary or trinary decision as to whether a biological tissue is healed without requiring the use of a radiographically opaque scale.

The gain mechanism may comprise a pin or roller in slot design, where the slot is at an angle with respect to the screw axis so that vertical motion is converted into horizontal motion along the slot with a gain defined by the relative slope of the slot with respect to the screw axis. Alternatively, scissors mechanisms can be used to convert the vertical to horizontal motion, with additional stages of the scissors providing additional gain. Alternatively, rack and pinion mechanisms may also be used. Other gain mechanisms including, telescoping, pulley, hydraulic, magnetic, geared, leveraged and other mechanisms are also known.

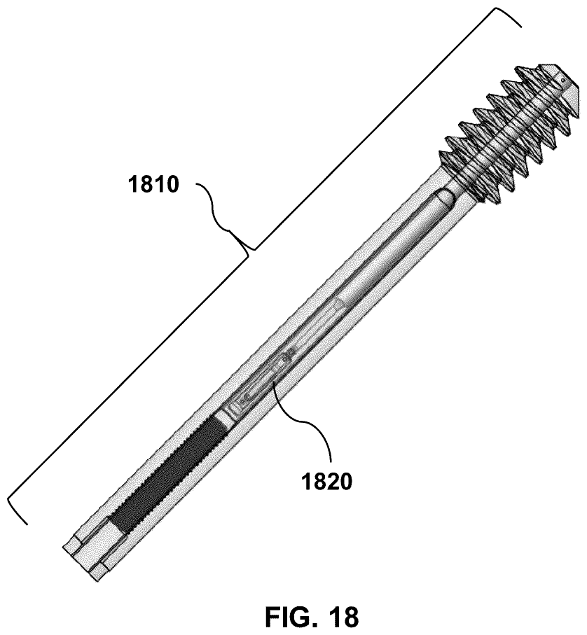

A sensor can be formed as a unitary part of an orthopedic fixation device at manufacture or can be mounted to a pre-existing orthopedic fixation device. For instance, a sensor can be added on to a fixation device following formation via any suitable attachment means. By way of example, the embodiment of FIG. 18 depicts a lag screw 1810 comprising an internal cannula with a strain sensor 1820 disposed therein. The lag screw may be formed as part of a dynamic hip screw as is shown in FIG. 15. Thus, the strain sensor and chemical sensor of the present disclosure may be used in combination. When a load non-parallel with the major axis of lag screw 1810 is applied to the head of lag screw 1810, bending may occur in the lag screw 1810. However, it is within the scope of the present disclosure that a load may be placed parallel to the major axis of the lag screw 1810, or strain sensor in general. In such an instance, a gain mechanism may be adopted from the similar mechanism used in the chemical sensor described above.

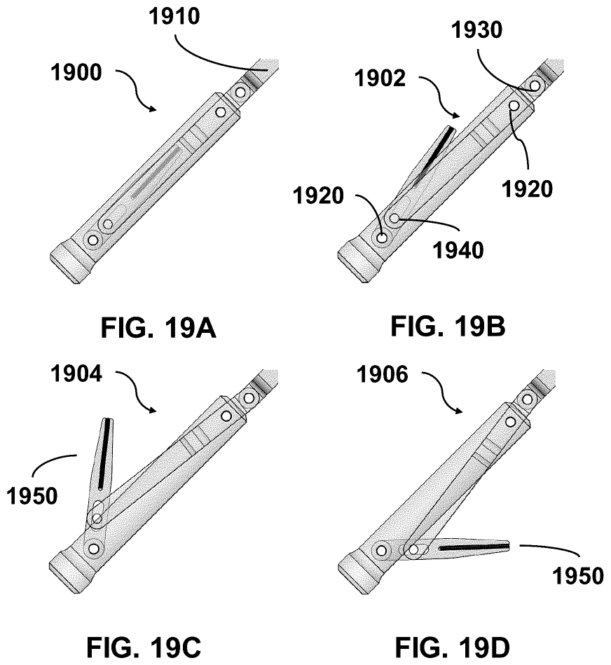

While bending may occur in the lag screw 1810, or any compliant orthopedic device or biological tissue subject to non-parallel loading, it may occur at a very small scale. Contemplated herein are mechanisms to amplify any bending motion that may be observed in a compliant orthopedic device or biological tissue. As shown in FIGS. 19A-19D, a non-parallel load applied to strain sensor 1900 causes gain mechanism 1950 to move outside of the body of strain sensor 1904, 1906. In this specific embodiment, a lever system utilizing dynamic pivot 1930, fixed pivots 1920 and sliding pinion 1940 allow for the amplification of an input force, as is demonstrated by the displacement of gain mechanism 1950. Gain mechanism 1950 may comprise a radiopaque marker, the movement of which can be visualized by a clinician.

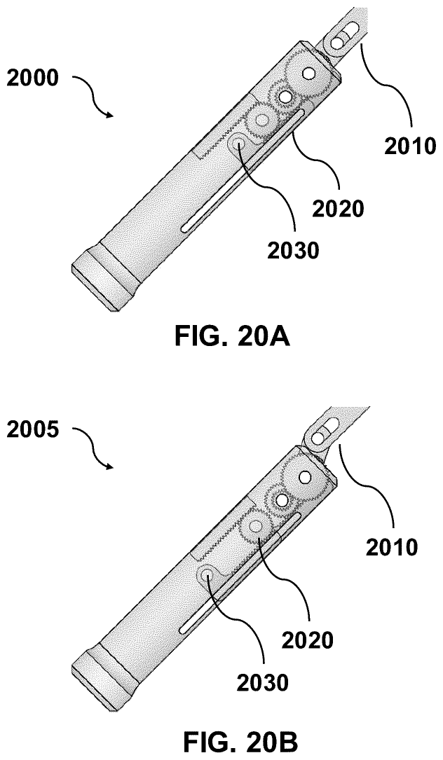

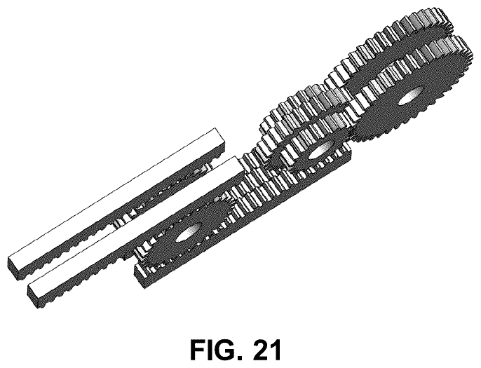

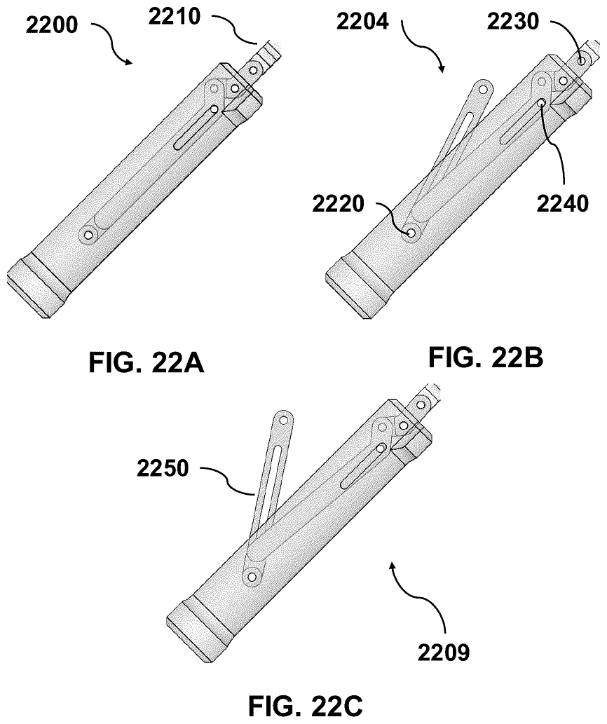

Other such gain mechanisms are envisioned. For instance, FIGS. 20A and 20B shows a strain sensor 2000 comprising a pickup arm 2010 that is operatively coupled to a gear system 2020 comprising a radiopaque marker 2030. As can be seen in the strain sensor 2005, application of a bending force causes a displacement of pickup arm 2010 which is translated by gear system 2020 into displacement of a radiopaque marker 2030. FIG. 21 provides a top down view of one potential gear system contemplated by the present disclosure. FIGS. 22A-C depict a strain similar in function to that of the strain sensor shown in FIGS. 19A-D. Through application of a force to the pickup arm 2210 of the strain sensor 2204, gain mechanism 2250 may be displaced through the amplification enabled by the lever system comprising the dynamic pivot 2230, fixed pivot 2220 and sliding pinion 2240.

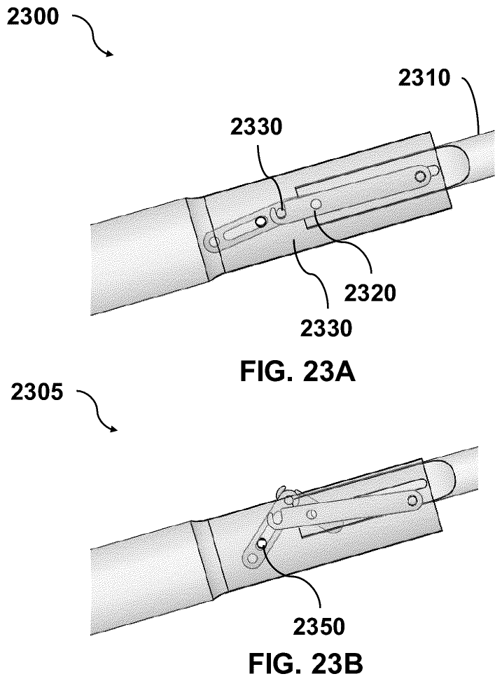

FIGS. 23A and 23B depict an exemplary strain sensor as described herein. For instance, the strain sensor 2300 comprises a pickup arm 2310 operatively coupled to a gain mechanism 2330 through a pivot 2320. As shown in strain sensor 2305, a movement of pickup arm 2310 causes a movement in gain mechanism through movement along sliding pinion 2350, thereby causing an exaggerated movement in radiopaque marker 2330.

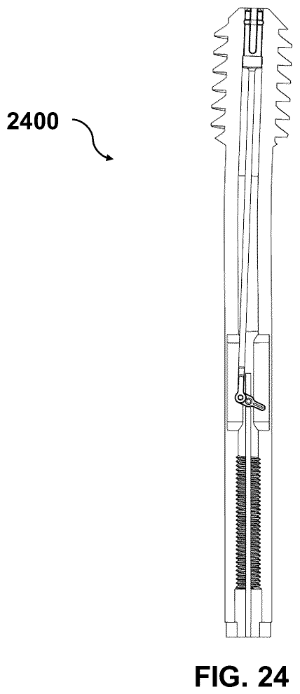

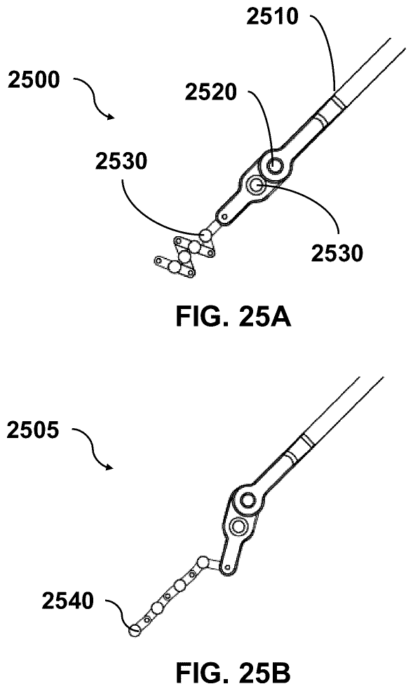

FIG. 24 depicts a lag screw comprising a strain sensor disposed within a cannula of said lag screw. FIGS. 25A and 25B provide a closeup view of the strain sensor disposed within the lag screw. Strain sensor 2500 comprises a sliding pivot 2520 and fixed pivots 2530. When a force is applied to the pickup arm 2510, the force is transduced through sliding pivot 2520 to cause the gain mechanism comprising a radiopaque marker 2540 to change conformations from a single-arm scissor to a straight conformation.

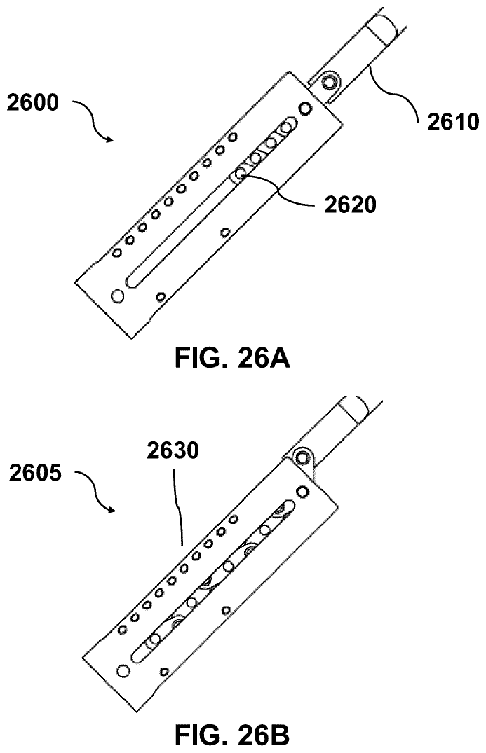

FIGS. 26A and 26B depict a strain sensor, such as the one depicted in FIGS. 26A and 26B which comprises a radiopaque marker 2620 sliding within casing 2630. Housing 2630 may optionally comprise radiopaque markers as a scale. As shown by the relative movement of radiopaque marker 2620, force applied to pickup arm 2610 causes the gain mechanism to move with parallel displacement.

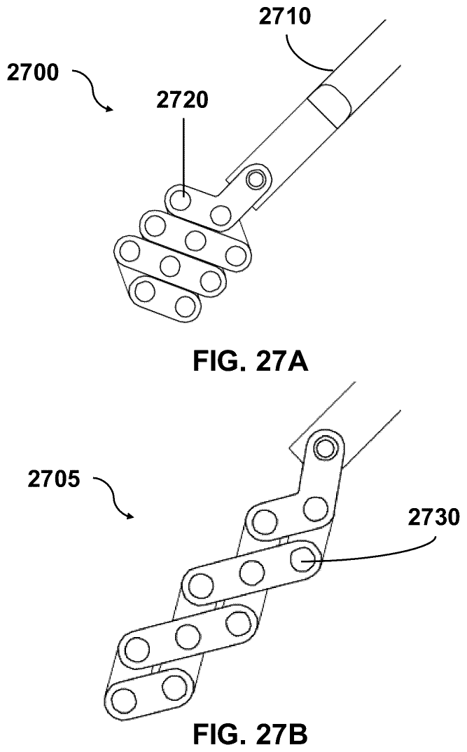

FIGS. 27A and 27B depict a dual-arm scissor gain mechanism 2700. Said gain mechanism 2700 comprises a fixed pivot 2720 which, when a force is applied to pickup arm 2710, causes gain mechanism 2705 to straighten, as can be visualized by radiopaque marker 2730.

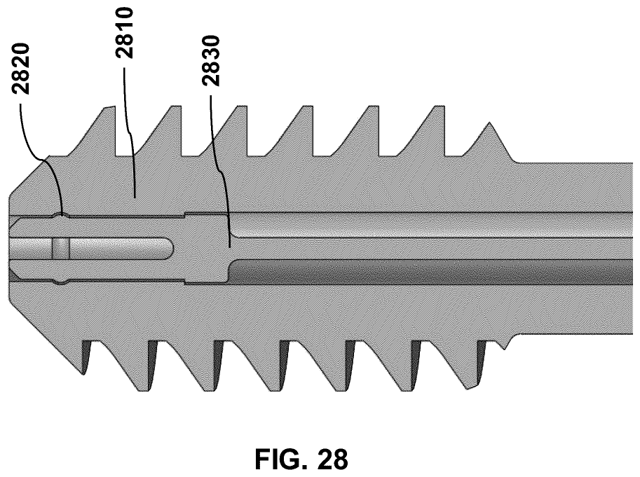

FIG. 28 depicts the tip of a lag screw with a portion of a strain sensor, particularly the pickup arm 2830, disposed within a cannula of the screw. The pickup arm 2830 interfaces with the screw 2810 at expansion ring 2820. While FIG. 28 depicts one means for securing the pickup arm to the cannula of the screw, it is within the ability of one of skill in the art to envision other means for such securement.

Use of disclosed strain sensors can reduce complications from refracture or hardware failure by restricting weight bearing until a bone is sufficiently healed. The quantitative metric within the devices can identify patients with delayed and non-union fractures so that adjunctive therapies may be used. The sensors can also provide information such that normally-healing patients can begin weight bearing when a fracture sufficiently heals, generally earlier than current treatment protocols. These capabilities can result in reduced healthcare costs, earlier return to work, and improved quality of life.

There is no particular arrangement of components or mounting orientation required for a sensor. In general a distal end and a proximal end of the strain sensor may be aligned adjacent to or inside of an orthopedic tissue, wherein the orthopedic tissue comprises a break disposed between the distal end and proximal end of the strain sensor. The strain sensor may be installed adjacent to or within the orthopedic tissue. One such means may comprise inserting the strain sensor within an orthopedic tissue.

As stated above, the strain sensor may be disposed within an orthopedic tissue, such as within a orthopedic implant disposed within the orthopedic tissue. For instance, the sensor may be disposed within the cannula of an implant as described above, such as within the cannula of a cannulated orthopedic screw such as used in a dynamic hip screw or cephalomedullary nail. The strain sensor may be placed in a cannula such that one end comprising the pickup arm may be coupled to an internal feature in the cannula of a screw. Additionally, the end of the strain sensor comprising the gain mechanism may have a similar means of attaching to the cannula of the screw, the two ends of the strain sensor being coupled by a pivot therebetween. Such interfaces may serve to secure the strain sensor in place within the cannula of a screw. Cannulas may be designed around guide wires which usually come in standard sizes, thus, sensors can be designed to fit a many manufactured cannulated screws based on the cannulation diameter. Contact with reproducible positioning and angle is needed between the screw and the strain sensor to detect bending of the screw with minimal device to device variation. This requires tools to grip the strain sensor, and position and orient to engage it within a cannula of a screw. Such a tool may comprise a sleeve that can compress an interface of the strain sensor, said interface designed to form a pinned fit, snap fit, or friction fit with the cannula of the screw.

Such a placement of the strain sensor within a cannula of a screw allows for the sensor to not impinge on any biological tissue it might otherwise should it be mounted on an exterior surface of a dynamic hip screw. Cannulated screws are typically placed using a guide wire, which is removed after screw placement; the cannulation is usually left empty and provides a space through which a strain sensor device can be added. The strain sensor may be installed by enclosing the strain sensor within a cartridge, wherein the cartridge compresses the strain sensor. The cartridge may then be inserted into an orthopedic implant, at which time the cartridge may decompress the strain sensor and be removed from the orthopedic implant. The strain sensor may, before or after removal of the cartridge, interface with a portion or a feature of the orthopedic implant.

When the screw does not bend much (e.g., at later stages of fracture healing) or for shorter screws or fractures stabilized with additional hardware, motion of a single pickup arm in the cannulation will be small and relatively hard to detect. Gain mechanisms can amplify these small changes so they are more easily observed. Although it is possible to attach gain mechanisms to the pickup arm which contacts the wall of the cannula when the pickup arm moves during bending, another highly convenient design is to have a separate gain mechanism cartridge that engages with the screw near the free end of the pickup arm and responds to displacement between it and the pickup arm. This requires the cartridge to have an expandable or sliding fit with the screw. To facilitate placement during surgery after the guidewire is removed, the cartridge can be attached to the pickup arm with a sleave or similar design, or both devices can be held with a placement tool, or the two devices can be placed sequentially.

The pickup arm and gain cartridge arrangement can also be used in other orthopedic devices in addition to screws, including plates, rods, and cages.

The strain sensor, orthopedic implant and optionally installation hardware may be provided to a clinician as a kit. Alternatively, the strain sensors of the present disclosure may be adapted to interface within standard orthopedic implants with no requirement for a modified orthopedic implant.

Further, the strain sensor may be installed with the aid of radiographic examination. For instance, radiographic examination may be useful when installing a strain sensor within the cannula of screw. After installation, the orthopedic implant or orthopedic tissue comprising the strain sensor may be subjected to a load to test the success of installation.

While certain embodiments of the disclosed subject matter have been described using specific terms, such description is for illustrative purposes only, and it is to be understood that changes and variations may be made without departing from the spirit or scope of the subject matter.

What is claimed is:

1. A strain sensor comprising:
a pickup arm comprising a free end and a static end, wherein the static end is directly or indirectly coupled to an orthopedic implant or orthopedic tissue;
a gain mechanism comprising a free end and a static end, wherein the static end of the gain mechanism is directly or indirectly coupled to the orthopedic implant or orthopedic tissue; and
wherein the free end of the gain mechanism and the free end of the pickup arm are operatively coupled.

2. The strain sensor of claim 1, wherein the gain mechanism comprises a scissor mechanism.

3. The strain sensor of claim 1, wherein the movement of the gain mechanism has a parallel displacement.

4. The strain sensor of claim 1, wherein the gain mechanism comprises a radiopaque marker.

5. The strain sensor of claim 1, wherein the orthopedic implant comprises the strain sensor.

6. The strain sensor of claim 1, wherein the strain sensor further comprises a scale comprising radiopaque markers.

7. The strain sensor of claim 1, wherein the static end of the pickup arm or the gain mechanism is secured to the orthopedic implant or orthopedic tissue by a friction fit, a snap fit or a pinned fit.

8. The strain sensor of claim 1, wherein the pickup arm and the gain mechanism are coupled by a pivot.

9. A strain-sensing orthopedic implant comprising the strain sensor of claim 1 coupled to a screw or a plate.

10. The strain-sensing orthopedic implant of claim 9, wherein the strain sensor is disposed within a cannula of the screw.

11. A method for installing a strain sensor, the method comprising:
aligning a distal end and a proximal end of the strain sensor of claim 1 adjacent or within an orthopedic tissue, wherein the orthopedic tissue comprises a break disposed between the proximal end and the distal end of the strain sensor.

12. The method of claim 11, wherein the strain sensor is disposed within a screw disposed within an orthopedic tissue, the strain sensor installed within the screw by enclosing the strain sensor in a cartridge which compresses the strain sensor, inserting the cartridge into the screw, decompressing the cartridge and removing the cartridge from the screw.

13. The method of claim 12, wherein the strain sensor interfaces with the screw after the cartridge is removed.

14. The method of claim 12, wherein the installation of the strain sensor is viewed under radiographic examination.

15. The method of claim 11, wherein the strain sensor is secured to an orthopedic tissue with a plate and screws.

16. The method of claim 11, wherein the orthopedic tissue comprises a femur or a tibia.

17. The method of claim 12 further comprising applying a load to the orthopedic tissue after removing the cartridge from the screw.

18. A kit comprising the strain sensor of claim 1 and an orthopedic implant.

19. The kit of claim 18, wherein the strain sensor is integrated into the orthopedic implant.

20. The kit of claim 18, further comprising installation hardware.
